(19) United States
(12) Patent Application Publication
(10) Pub. No.: US 2023/0031757 A1
(43) Pub. Date: Feb. 2, 2023

(54) HEALTH APPLICATION

(71) Applicant: MYTABOLITE, INC., Irvine, CA (US)

(72) Inventors: Steven N. BRONSON, Laguna Beach, CA (US); James Raymond HILSER, Los Angeles, CA (US)

(73) Assignee: MYTABOLITE, INC., Irvine, CA (US)

(21) Appl. No.: 17/815,888

(22) Filed: Jul. 28, 2022

Related U.S. Application Data
(60) Provisional application No. 63/226,674, filed on Jul. 28, 2021.

(51) Int. Cl. G16H 50/20; G16H 40/40; G16H 50/30

(52) U.S. Cl. CPC G16H 50/20; G16H 40/40; G16H 50/30

(57) ABSTRACT

A method may include obtaining external health data corresponding to other people. The method may also include training a predictive model using the external health data the predictive model generates an association between personal health data related to a user and the external health data. In addition, the method may include obtaining the personal health data. Further, the method may include comparing the personal health data to the predictive model. The method may include generating a risk report based on the comparison, the risk report indicating a potential health risk of the user.

### CROSS-REFERENCE TO RELATED APPLICATION

This patent application claims the benefit of and priority to U.S. Provisional App. No. 63/226,674 filed Jul. 28, 2021, titled “HEALTH APPLICATION,” which is incorporated in the present disclosure by reference in its entirety.

### FIELD

The embodiments discussed in the present disclosure are related to a health application.

### BACKGROUND

Unless otherwise indicated herein, the materials described herein are not prior art to the claims in the present application and are not admitted to be prior art by inclusion in this section.

Personal health data (e.g., health data of a person) may be difficult for a person to obtain and may be equally difficult for the person to interpret. The personal health data may include health indicators that are derived from blood tests such as, a comprehensive metabolic panel or a lipid panel. Specific biomarker levels derived from such panels may include, but are not limited to, low-density lipoprotein, hemoglobin A1C, and gamma-glutamyl transferase. Differential levels of these biomarkers may be indicative of diseases and have the potential to be leveraged to gain greater insight in a person's health. Therefore, in some circumstances, knowledge about the personal health data, including potential health risks, may lead the person to make choices which may improve their health and lessen identified risks.

The subject matter claimed in the present disclosure is not limited to embodiments that solve any disadvantages or that operate only in environments such as those described above. Rather, this background is only provided to illustrate one example technology area where some embodiments described in the present disclosure may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an embodiment, a method may include obtaining external health data corresponding to other people. The method may also include training a predictive model using the external health data the predictive model generates an association between personal health data related to a user and the external health data. In addition, the method may include obtaining the personal health data. Further, the method may include comparing the personal health data to the predictive model. The method may include generating a risk report based on the comparison, the risk report indicating a potential health risk of the user.

The objects and advantages of the embodiments will be realized and achieved at least by the elements, features, and combinations particularly pointed out in the claims.

Both the foregoing general description and the following detailed description are given as examples and are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 illustrates a block diagram of an example environment of a health application;

FIG. 2 illustrates an example method associated with a health application;

FIG. 3 illustrates a flowchart of a method of generating a risk report; and

FIG. 4 illustrates a block diagram of an example computing system,

all according to at least one embodiment described in the present disclosure.

### DETAILED DESCRIPTION OF EMBODIMENTS

Personal health data (e.g., health data of a person) may be difficult for a person to obtain and may be equally difficult for the person to interpret. The personal health data may include health indicators that are derived from blood tests such as, a comprehensive metabolic panel or a lipid panel. Specific biomarker levels derived from such panels may include, but are not limited to, low-density lipoprotein, hemoglobin A1C, and gamma-glutamyl transferase. Differential levels of these biomarkers may be indicative of diseases and have the potential to be leveraged to gain greater insight in a person's health. In some circumstances, knowledge about the personal health data, including potential health risks, may lead the person to make choices which may improve their health and lessen identified risks.

In some circumstances, the person may struggle to know and/or understand the personal health data (e.g., their overall health, health of specific organs, test results, etc.). The person may be unaware of potential health risks until they experience an acute injury, an organ failure, and/or other problems, which often occur at a point in time in which mitigation of the health risk is more difficult. In some circumstances, the person may seek to maintain good health and may obtain health tests, such as a blood test, to increase their personal health data. In some instances, test results (e.g., results of the health tests) may be complex and/or replete with numbers, which may not provide understandable insight to the person as to their current health or where issues may arise at a future time.

A person in a supervisory position (e.g., a supervisor, a manager, a boss, a principal, a teacher, etc. (generally referred to in the present disclosure as the supervisor)) may struggle to know and/or understand potential health risks that may potentially impact people within their supervision (e.g., employees, students, etc. (generally referred to in the present disclosure as the group)). For example, the supervisor may be unaware that the group is more prone to certain health issues such as diabetes, heart conditions, respiratory conditions, etc., which may impact operations of a facility (e.g., a warehouse, an office, a factory, an enterprise, a division, a school, etc.) at which the group works at. In addition, the supervisor may be unaware of practices or actions that may improve the health of the group, which may improve efficiency of the facility.

In some embodiments, a computing device may include a health application that determines health indicators of the person and/or the group based on corresponding personal health data and external health data. For example, the health application may determine the health indicators based on test results of a blood test or other health tests and the external health data. The health indicators may include biomarker data and/or other health indicators of the person and/or the group.

In some embodiments, the health application may obtain the external health data (e.g., health data of other people) from large sets of health data and/or associations created between health indicators of the other people. The health application may train (e.g., develop) a predictive model using a machine learning (ML) algorithm and the external health data. The predictive model may be trained to identify associations between the personal health data and the external health data.

The health application may use the predictive model to identify potential health risks of the person and/or the group based on the corresponding health indicators and the external health data. In addition, the health application may provide mitigation recommendations (e.g., recommendations to improve and/or mitigate the potential risks to the health of the person and/or the group) based on the identified potential health risks. The health application may determine the potential health risks and present the mitigation recommendations based on the health indicators and the external health data. In some embodiments, the potential health risks identified by and/or the mitigation recommendations provided by the health application may contribute to a better understanding of the health of the person and/or the group.

The health application may provide a risk report based on the analysis of the personal health data and the external health data using the predictive model. The risk report may include the potential health risks of the person and/or the group. In addition, the risk report may include a condensed assessment of the personal health data (e.g., the health indicators) in a format that is simple to understand by the person and/or the group. Further, the risk report may indicate if the person and/or the group are in a high-risk category for a health category (e.g., diabetes, heart conditions, etc.). The health application may provide the risk report to the person, the group, the supervisor, or some combination thereof based on the analysis. Alternatively or additionally, the health application may provide the risk report including the mitigation recommendations to help combat the identified potential health risks, which may contribute to an increased wellbeing of the person and/or the group.

In some embodiments, the health application may determine an efficient number of health tests to administer within the group to determine a prevalence of a corresponding health condition based on other health indicators (e.g., a weight, a height, a resting heart rate, a gender, a waist circumference, a blood pressure, a cholesterol level, a blood sugar level, and/or other measurable health indicators of the people within the group). For example, the health application may use the predictive model and the other health indicators of the group to determine a likelihood of a percentage of the people within the group experiencing a heart condition. In addition, the health application may determine the efficient number of health tests to administer within the group to determine if the people within the group experience the heart condition at the expected percentage. The risk report may indicate the efficient number of health tests to administer within the group.

The health application may increase a rate and/or speed at which the personal health data of a large number of people (e.g., the group of people may include thousands, hundreds of thousands, or more people) can be analyzed and mitigation recommendations provided for. For example, the health application increases a size of the group that can receive the risk report within a particular period of time. In addition, the health application may permit doctors and/or health care professionals to make a determination about the group without having to examine each person within the group. Further, the health application may, based on the risk report, cause changes within an environment or health care program to occur to improve the health of the person and/or the group. For example, the health application may schedule or cause to be scheduled breaks during a period of time for the group to exercise, consume healthier food, consume snacks that improve a specific aspect of the health of the group, etc. The health application may operate in parallel with supervision by medical professionals not in lieu of medical professionals.

These and other embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise.

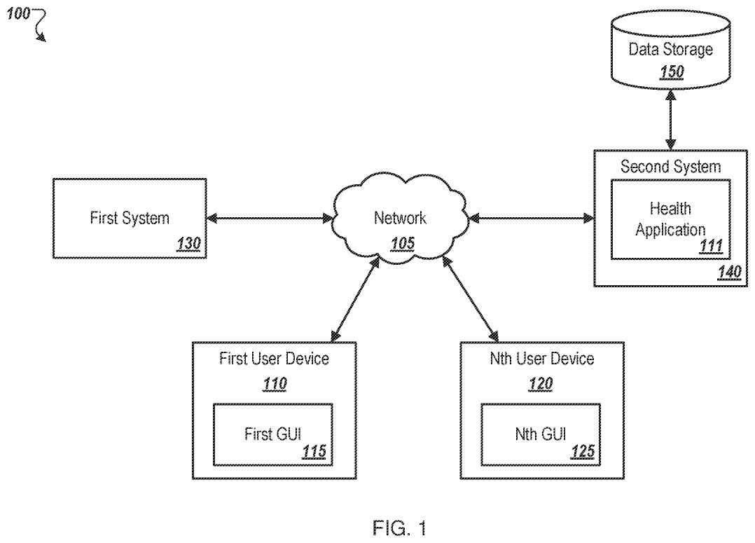

FIG. 1 illustrates a block diagram of an example environment 100 of a health application 111, in accordance with at least one embodiment described in the present disclosure. The environment 100 may include a network 105, a first user device 110, an nth user device 120, a first system 130, a second system 140, and a data storage 150. The first user device 110 may include a first graphical user interface (GUI) 115. The nth user device 120 may include an nth GUI 125.

In some embodiments, the network 105 may communicatively couple the first user device 110, the nth user device 120, the first system 130, and/or the second system 140. In some embodiments, the network 105 may be any network or configuration of networks configured to send and receive communications between systems. In some embodiments, the network 105 may include a wired network, an optical network, and/or a wireless network, and may include numerous different configurations, including multiple different types of networks, network connections, and protocols to communicatively couple systems in the environment 100.

In some embodiments, the first user device 110 may include any electronic or digital computing device and/or system. For example, the first user device 110 may include a desktop computer, a laptop computer, a smart phone, a smart watch, a mobile phone, a tablet computer, and/or any other computing device that may be used for displaying information and/or receiving interactions from a user (e.g., the person and/or a person within the group).

In some embodiments, the first user device 110 may include a memory and at least one processor (not illustrated in FIG. 1), which are configured to perform operations as described in this disclosure, among other operations. In some embodiments, the first user device 110 may include computer-readable instructions stored in the memory that are configured to be executed by the first user device 110 to perform operations described in this disclosure.

In some embodiments, the first user device 110 may operate the first GUI 115. Operating the first GUI 115 may include executing instruction to cause the display of the first GUI 115, to receive inputs from the user (e.g., the person or a person within the group), and to generate and display outputs. In some embodiments, the instructions to operate the first GUI 115 may be stored in the memory of the first user device 110.

In some embodiments, the first GUI 115 may receive user input that may be processed and/or transmitted by the first user device 110. For example, the first GUI 115 may receive user input directing the first user device 110 to request appointment information from the first system 130 over the network 105. Alternatively or additionally, the first GUI 115 may display information and/or data that may be responsive to a request received through the first GUI 115 and transmitted by the first user device 110. For example, the first GUI 115 may display personal health data, a health indicator, a test result, a health recommendation, a mitigation recommendation, a risk report, etc. following the user obtaining a health test and requesting associated test results (e.g., personal health data).

In some embodiments, the nth user device 120 may be analogous to the first user device 110 and may be representative of any number of users in the environment 100 (e.g., any number of people within the group). Alternatively or additionally, the nth GUI 125 may be analogous to the first GUI 115 in that the nth GUI 125 may provide an interface to the user to receive inputs and/or to display health data and/or information. In some embodiments, the first user device 110 through the nth user device 120 may be associated with a facility in which the group works or otherwise engages.

In some embodiments, the first system 130 may be any electronic or digital computing device and/or system. For example, the first system 130 may include a desktop computer, a laptop computer, a smartphone, a mobile phone, a tablet computer, server, a processing system, or any other computing device that may be used for performing some or all of the operations described in this disclosure and for communicating data between the first system 130 and the first user device 110, the nth user device 120, and/or the second system 140.

In some embodiments, the first system 130 may be associated with a test facility, such as a blood test facility that may administer blood tests and/or determine associated test results (e.g., blood test results). In some embodiments, the first system 130 may be located at the associated test facility. Alternatively or additionally, the first system 130 may be remote from the associated test facility and may transfer data to and from the associated test facility.

In some embodiments, the first system 130 may receive user requests over the network 105, such as from the first user device 110 and/or the nth user device 120. For example, the user requests may include a request for appointment availability (e.g., available dates and times for an appointment), a request for an appointment reservation, a request for a test result status, a request for a risk report, and/or another request. Alternatively or additionally, the first system 130 may receive data requests over the network 105, such as from the second system 140. For example, the data requests may include a request for external health data, a request for the personal health data, a request for the biomarker data, and/or a request for other health measurements that may be associated with the test results.

In some embodiments, the first system 130 may transmit data and/or information over the network 105, such as to the first user device 110, the nth user device 120, and/or the second system 140. For example, the first system 130 may transmit an appointment reminder, a test status update, an appointment action (e.g., how to prepare for a test administration), the potential health risk, the mitigation recommendation, the risk report, etc., to the user, such as via the first user device 110. In another example, the first system 130 may transmit the personal health data, the external health data, the biomarker data, longitudinal biomarker data, and/or the health indicators (e.g., weight, height, resting heart rate, etc.) to another system, such as the second system 140.

In some embodiments, the second system 140 may be any electronic or digital computing device and/or system. For example, the second system 140 may include a desktop computer, a laptop computer, a smartphone, a mobile phone, a tablet computer, a server, a processing system, or any other computing device that may be used for performing some or all of the operations described in this disclosure and for communicating data between the second system 140 and the first user device 110, the nth user device 120, the first system 130, and/or the data storage 150.

In some embodiments, the data storage 150 may be communicatively coupled to the second system 140 such that health data (e.g., the personal health data, the external health data, or some combination thereof) may be transferred between the second system 140 and the data storage 150. In some embodiments, the communicative coupling between the data storage 150 and the second system 140 may include any configuration of networking elements configured to send and receive communications therebetween. For example, the communications between the second system 140 and the data storage 150 may include peripheral component interconnect (PCI), PCI Express, Ethernet, wireless transfer such as Bluetooth®, Wi-Fi, WiMAX, cellular communications, and/or other methods of communication.

In some embodiments, the data storage 150 may include computer-readable storage media such as Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store data or data structures which may be accessed by a general-purpose or special-purpose computer.

In some embodiments, the second system 140 may include the health application 111. The health application 111 may include code and routines configured to enable the second system (e.g., a computing device) 140 to perform one or more operations with respect to identifying the potential health risk, determining the mitigation recommendation, generating the risk report, or any other operation described in the present disclosure. Additionally or alternatively, the health application 111 may be implemented using hardware including a processor, a microprocessor (e.g., to perform or control performance of one or more operations), a field-programmable gate array (FPGA), or an application-specific integrated circuit (ASIC). In some other instances, the health application 111 may be implemented using a combination of hardware and software. In the present disclosure, operations described as being performed by the health application 111 may include operations that the health application 111 may direct a corresponding system (e.g., the second system 140, the first system 130, or some combination thereof) to perform.

The health application 111 may obtain the personal health data and/or the external health data from one or more sources. For example, the health application 111 may obtain the personal health data over the network 105 from the user, such as from the user of the first user device 110 and/or the user of the nth user device 120, or from other systems and devices, such as the first system 130.

In some embodiments, the second system 140 may transmit and/or receive the personal health data to/from the data storage 150. For example, in instances in which the second system 140 obtains the personal health data from the first user device 110, the second system 140 may transmit the personal health data to the data storage 150. In these and other embodiments, the second system 140 may transmit and/or receive the external health data from the data storage 150. For example, in instances in which the second system 140 obtains the external health data from the first system 130, the second system 140 may transmit the external health data to the data storage 150.

In some embodiments, the health application 111 may analyze the personal health data using the predictive model. For example, the health application 111 may generate associations between one or more elements of the personal health data (e.g., portions of the biomarker data, the longitudinal biomarker data, the other health indicators, etc.) and/or determine relationships between one or more elements of the personal health data. In some embodiments, the personal health data may be obtained from the user, another system, data storage, and/or other locations. For example, the personal health data may be obtained from the user of the first user device 110, the first system 130, and/or the data storage 150.

The health application 111 may train a predictive model using the external health data and a ML algorithm. In some embodiments, the predictive model may determine associations between health data (e.g., the biomarker data) within the external health data. For example, the predictive model may determine that the presence of certain biomarkers is associated with other health indicators. Alternatively or additionally, the predictive model may develop associations between different portions of the external health data based on an observed level of biomarkers in the biomarker data.

The health application 111 may develop the predictive model based on the external health data and/or based on the analysis of the external health data. For example, the predictive model may be trained to determine that a presence and/or a threshold quantity of one or more elements of the external health data correspond to a prediction relative to other elements of external health data. (e.g., imputation of biomarker levels and quality control (QC) of test results). For example, if a level of one or more of the biomarkers within the external health data is known, the health application 111 may determine a level of one or more biomarkers that are not known based on the known levels of the one or more biomarkers. The health application 111 may use the determined level of the one or more biomarkers that were not known to perform QC data of the personal health data (e.g., determine if there are anomalies in the personal health data). In some embodiments, the health application 111 may double check hard results by comparing an imputed level of a biomarker to a real level of the biomarker. For example, without the health application 111, a physician may tell the user to retake a blood test because the doctor feels a biomarker level in the personal health data (e.g., the test result) may be wrong. However, the health application 111 may determine whether an actual anomaly is present in the personal health data (e.g., the test result) or if the issue is an artifact of the personal health data.

The predictive model may use a training a set of health data (e.g., a known health data set) to determine levels of biomarkers that are associated with a disease outcome. Using ML techniques such as weighted measures and repeated sampling, the predictive model may be trained to include algorithms to determine what combination of biomarker levels corresponds to an “unhealthy” individual. Furthermore, given the plethora of blood tests available, the algorithms of the predictive model may use common and/or inexpensive blood tests, to generate results. Accordingly, variable levels that are trained into the predictive model may be what the health application 111, via the predictive model, uses to determine if the user is unhealthy or not. For example, the predictive model may utilize coronary artery disease (CAD) and/or type II diabetes (T2D) as surrogates to determine whether the user is “unhealthy” or not. Therefore, the health application 111 may determine that the user is considered high risk if the user fits the ML algorithms within the predictive model for CAD or T2D and may notify the user of such. The health application 111 may also determine that the user is considered high if the user fits the ML algorithms within the predictive model for chronic kidney disease and/or non-alcoholic fatty liver disease. In some embodiments, the risk report generated by the health application 111 may not serve as a diagnostic machine for CAD or T2D. Rather, the risk report may be generated and the user may bring the risk report to a physician or health care provider. This may permit the health application 111 to operate in parallel with the current clinical infrastructure, not in lieu of.

Alternatively or additionally, the health application 111 may provide an interpretation of the personal health data. For example, the health application 111 may make determinations regarding likely health outcomes (e.g., likelihood of the user experiencing a health issue) in view of the personal health data using the predictive model.

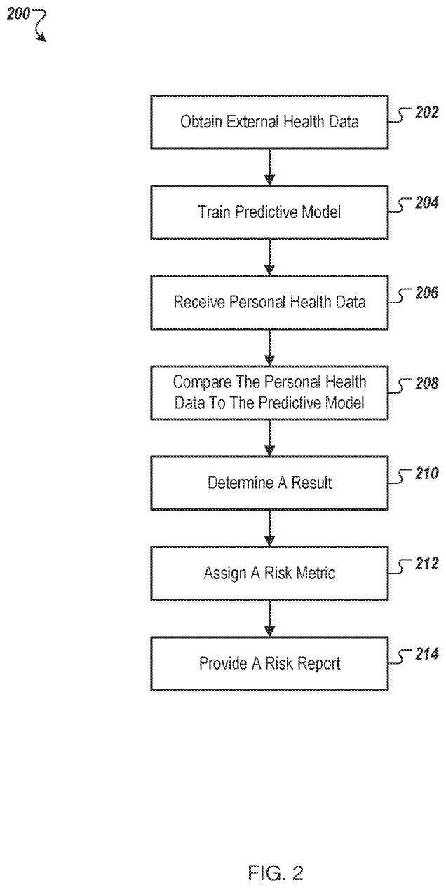

In these and other embodiments, the health application 111 may implement a method of obtaining the external health data, analyzing the external data, developing the predictive models associated with the external health data, obtaining the personal health data, comparing the personal health data to the predictive models, and/or determining mitigation recommendations and/or generating a risk report based on the comparison. The method may be further described relative to the method 200 of FIG. 2.

In some embodiments, the data storage 150 may store the personal health data, the external health data, the biomarker data, the longitudinal biomarker data, the risk report, or some combination thereof. In these and other embodiments, the personal health data may include the biomarker data. In some embodiments, the biomarker data may include measurable health indicators associated with a biological state or condition of the user, which, in some instances, may be based on blood samples. In some embodiments, the health application 111 may receive the personal health data from the first system 130 and/or stored in the data storage 150. Alternatively or additionally, the health application 111 may receive the personal health data from the first user device 110 and/or the nth user device 120 and/or stored in the data storage 150. Alternatively or additionally, the health application 111 may acquire the personal health data from other sources, including other systems and/or other data storage devices.

In some embodiments, the biomarker data stored in the data storage 150 may be associated with other health indicators. For example, the biomarker data of the user at a first time may be associated with other health indicators obtained from the user at the first time. In some embodiments, the other health indicators may include a height, a weight, a gender, a resting heart rate, a waist circumference, a blood pressure, a cholesterol level, a blood sugar level, and/or other measurable health indicators of the user. In some embodiments, each instance of the biomarker data (e.g., each entry of the biomarker data in the data storage 150) may be linked with the associated other health indicators. For example, the biomarker data and the associated other health indicators from the user at the first time may be a unique data entry in the data storage 150 relative to the biomarker data and associated other health indicators from the user at a second time.

In these and other embodiments, the health application 111 may correlate the biomarker data with the other health indicators based on the source of the biomarker data and the time the biomarker data and other health indicators were obtained. For example, the health application 111 may associated the biomarker data obtained from the user at the first time with other health related aspects of the user as applicable at the first time, such as a health history related to the organs and/or organ systems of the user (e.g., heart health and cardiovascular disease, kidney health and chronic kidney disease, etc.).

In some embodiments, the personal health data and/or the external health data stored in the data storage 150 may be stored without personally identifying information. For example, the external health data in the data storage 150 may be unassociated with a specific person and may represent data points relative to the biomarker data and the other health indicators. In the present disclosure, the personal health data and/or the external health data may include the biomarker data, the other health indicators as provided above, and/or combinations thereof.

In some embodiments, the data storage 150 may store the personal health data including organ context data and/or organ system context data (collectively referred to in the present disclosure as organ context data) representative of a health status of an organ and/or an organ system. The organ context data may include functional and/or responsive details related to an organ such as varying functional levels of an organ, typical responses by an organ to an expected environment, conditions, or to different stimuli, organ performance in view of measured biomarker data related thereto, and/or other organ context data.

In some embodiments, the organ context data may be associated with the biomarker data. For example, in instances in which a portion of the biomarker data is present and/or a portion of the biomarker data is above a threshold value, a portion of the organ context data may be associated with the corresponding biomarker data and/or biomarker levels. For example, a high-density lipoprotein (HDL) level below a threshold value in conjunction with a total cholesterol level above a threshold value may be associated with heart health and a potential for heart issues as may be included in the heart context data.

In these and other embodiments, the health application 111 may associate the organ context data, the personal health data, the external health data, or some combination thereof with one another. For example, the health application 111 may retrieve a portion of the personal health data from the data storage 150 and may associate the personal health data with the organ context data. In some embodiments, the health application 111, using the predictive model, may make associations between the personal health data and the organ context data as the personal health data is received by the second system 140. Alternatively or additionally, the health application 111 may retrieve the personal health data from the data storage 150 and may make associations between the personal health data and the organ context data. Alternatively or additionally, the health application 111 may adjust and/or update existing associations of the personal health data and the organ context data as additional personal health data is received.

In some embodiments, the health application 111, using the predictive model, may analyze and/or make interpretations based on the personal health data. For example, after making associations between the personal health data and the organ context data, the health application 111 may generate the risk report (e.g., determine the potential health risks and/or the mitigation recommendation) to reduce the potential health risks associated with the user based on the personal health data. Additional details related to analyzing and/or interpreting the personal health data may be further discussed relative to the method 200 of FIG. 2.

FIG. 2 illustrates an example method 200 associated with the health application 111 of FIG. 1, in accordance with at least one embodiment described in the present disclosure.

The method 200 may be performed by the health application 111 including processing logic that may include hardware (circuitry, dedicated logic, etc.), software (such as is run on a general-purpose computer system or a dedicated machine), or a combination of both, which processing logic may be included in the first system 130 and/or the second system 140 of FIG. 1, or another computer system or device. However, another system, or combination of systems, may be used to perform the methods.

For simplicity of explanation, the method 200 is described herein is depicted and described as a series of operations. However, operations in accordance with this disclosure may occur in various orders and/or concurrently, and with other operations not presented and described herein. Further, not all illustrated operations may be used to implement the method 200 in accordance with the disclosed subject matter. In addition, those skilled in the art will understand and appreciate that the method 200 may alternatively be represented as a series of interrelated states via a state diagram or events. Additionally, the method 200 is capable of being stored on an article of manufacture, such as a non-transitory computer-readable medium, to facilitate transporting and transferring the method 200 to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 200 may begin at block 202 where the processing logic may obtain external health data. In some embodiments, the external health data may include data stored in a data storage, such as the data storage 150 of FIG. 1, that may have been previously obtained. Alternatively or additionally, the external health data may be obtained from user data that may be submitted by a user. Alternatively or additionally, the external health data may be obtained from a remote system.

In some embodiments, the external health data may include organ context data and/or personal health data, which may include the biomarker data and/or the other health indicators, as described in the present disclosure.

At block 204, the processing logic may train a predictive model using the external health data. In some embodiments, the predictive model may include associations between the biomarker data, the other health indicators, and/or the organ context data within the external health data. For example, the predictive model may determine that the presence of certain biomarkers in the biomarker data within the external health data is associated with the other health indicators and/or the organ context data within the external health data. Alternatively or additionally, the predictive model may develop associations between the biomarker data, the other health indicators, and/or the organ context data based on an observed level of biomarkers in the biomarker data within the external health data. For example, in instances in which a level of a first biomarker of the biomarker data within the external health data is greater than a threshold value and a particular organ status is present, the predictive model may associate the level of the first biomarker with the particular organ status, such that future indications of the level of the first biomarker may indicate the particular organ status is present or may be likely to become present.

In some embodiments, the processing logic may iteratively train the predictive model. For example, the processing logic may initially train the predictive model using initial external health data and the processing logic may adjust and/or update the predictive model as additional external health data is made available. In some embodiments, the additional external health data may include new personal health data from an existing user, personal health data from a new user, and/or external health data from a database not including the initial external health data. In some embodiments, the additional external health data may include new or changed biomarker data, new or changed other health indicators, or new or changed organ context data, with respect to each source of the additional external health data.

In these and other embodiments, the user may include a person operating a user device that may request a health test, such as a blood test, and receive test results related to the health test. Additionally or alternatively, the user may include a person within the group. The user may choose to transmit their personal health data to the system associated with the predictive model. For example, the user device may be analogous to the first user device 110 of FIG. 1 and the system associated with the predictive model may be analogous to the second system 140 of FIG. 1.

In some embodiments, after the predictive model is trained, the processing logic may validate the predictive model. The validation of the predictive model may occur at any time after the predictive model is trained. For example, the processing logic may validate the predictive model after the predictive model is updated with the additional external health data, and/or at any other time occurring after the predictive model is trained. In some embodiments, the validation of the predictive model may include comparing the results of the predictive model to a known set of health data. For example, after the predictive model is trained, the processing logic may cause the predictive model to analyze the known set of health data (e.g., a different set of health data than was used to train the predictive model) to determine an accuracy of the predictive model and/or to further tune the predictive model. In some embodiments, the processing logic may update the predictive model in view of the validation thereof. For example, in instances in which the predictive model misses a prediction by greater than a threshold value, the processing logic may update the predictive model to include the missed prediction in future predictive scenarios. Alternatively or additionally, in instances in which the predictive model may not yet be calibrated to make a prediction relative to a new portion of health data in the known set of health data, the processing logic may train the predictive model based on the new portion of the health data, such that the predictive model may make a prediction relative to the new portion of the health data in future predictive scenarios.

At block 206, the processing logic may obtain personal health data. In some embodiments, the personal health data may include health data indicators and may be obtained from the user, such as the user transmitting the personal health data to a system associated with the processing logic. For example, the user may operate a user device, such as the first user device 110, to submit the personal health data to the system associated with the processing logic. Alternatively or additionally, the processing logic may obtain the personal health data from a data storage, such as the data storage 150 of FIG. 1. In some embodiments, the personal health data may include the biomarker data, the longitudinal biomarker data, and/or the other health indicators such as gender, weight, height, resting heart rate, etc.

At block 208, the processing logic may compare the personal health data (e.g., the user health indicators) to the predictive model. For example, the processing logic may compare elements of the personal health data, such as biomarker inclusions and/or biomarker levels, to known biomarker inclusions or biomarker levels in the predictive model. In some embodiments, the processing logic may compare individual elements between the personal health data (e.g., user health indicators) and the predictive model. For example, the processing logic may obtain a first biomarker of the biomarker data within the personal health data and compare it to biomarker data in the predictive model. Alternatively or additionally, the processing logic may compare multiple elements of the personal health data to the predictive model. For example, the processing logic may obtain one or more biomarkers of the biomarker data, one or more longitudinal biomarkers, and/or the other health indicators within the personal health data to compare to the predictive model.

At block 210, the processing logic may determine a result based on the comparison between the personal health data and the predictive model. In some embodiments, the results may include associating the personal health data with one or more elements of the predictive model. For example, the processing logic may determine that the presence of two user health indicators within the personal health data above a threshold level may be associated with heart health of the predictive model, which may be associated with poor organ function, such as heart disease.

At block 212, the processing logic may assign a risk metric to the results. In some embodiments, the risk metric may include a likelihood that the user, whose user health indicators were compared to the predictive model, may experience health issues and may experience a medical intervention at a future time. In some embodiments, the risk metric may be associated by organ and/or organ systems. For example, the risk metric may provide an indication that an organ system may be underperforming and/or may be at risk for future conditions or issues. In some embodiments, the risk metric may be determined on a scale, such as a value between zero and one hundred. For example, in instances in which the processing logic compares the personal health data to the predictive model and determines the user's heart health is above a threshold (e.g., indicating good heart health), the processing logic may assign a lower value to the risk metric. Alternatively or additionally, the risk metric may include a discrete number of categories that may generally describe an associated organ's health. For example, the discrete categories may include, poor, fair, good, and/or excellent and the processing logic may assign user's risk metric into a group including one of the discrete categories based on the risk metric exceeding various threshold related to the discrete categories.

At block 214, the processing logic may provide a risk report to the user and/or the supervisor. The risk report may include mitigation recommendations based on the risk metric, the results, and/or the comparison of the personal health data to the predictive model. In some embodiments, the mitigation recommendations may include various actions the user may take to improve health related to the organs and/or the organ systems that were identified in the result determinations and/or risk assignment (e.g., block 210 and/or block 212, respectively). For example, the risk report may include mitigation recommendations that include a suggested change to a diet of the user, a suggested change to an activity level of the user (e.g., exercise), a suggested change to a sleep pattern of the user, and/or other recommendations that may be directed at improving the health of the user.

The risk report may include the mitigation recommendations for the supervisor to improve an environment of the group. For example, the mitigation recommendations may include a suggested schedule of breaks during a period of time for the group to exercise, consume healthier food, consume snacks that improve a specific aspect of the health of the group, etc. The risk report may also include an efficient number of health tests to administer within the group to determine a prevalence of a corresponding health condition within the group. For example, the mitigation recommendations may indicate testing twenty five percent of the group will be sufficient to properly determine if the group experience the health condition at the expected percentage.

In some embodiments, the processing logic may provide (e.g., transmit to an external device or display via a display screen) the risk reports (e.g., the results and/or the mitigation recommendations) to the user and/or the supervisor. In some embodiments, the risk report may include data divided into various organ and/or organ systems. For example, the processing logic may determine a set of the biomarker data and/or the other health indicators within the personal health data is related to heart health and the processing logic may transmit the risk report including the results and/or the mitigation recommendations as heart related data. Alternatively or additionally, the processing logic may transmit the underlying numerical data of the biomarker data and/or the other health indicators within the personal health data that may be related to the user and/or may be associated with a specific organs and/or organ systems.

Modifications, additions, or omissions may be made to the method 200 without departing from the scope of the present disclosure. For example, the method 200 may include more or fewer elements than those illustrated and described in the present disclosure.

In some embodiments, the processing logic may compare the personal health data including the user health indicators, the biomarker data, or some combination thereof to expected levels of health indicators within the external health data. The personal health data may be compared to upper and/or lower thresholds to determine if corresponding user health indicators fall within a statistically likely result. For example, the processing logic may compare first biomarker data within the personal health data to an associated expected biomarker data level within the external data and/or the predictive model, which may enable the processing logic to determine whether the biomarker data within the personal health data is likely from an accurate test. In some embodiments, determining the accuracy of the personal health data may contribute to determining the accuracy of a health test, such as a blood test, and may reduce or remove occurrences of additional testing in instances in which the test results may be questioned as unusual or faulty.

In some embodiments, the processing logic may develop additional predictive models related to the personal health data. For example, the processing logic may develop a product marketing model that may associate biomarker data and/or other health indicators with a likelihood the associated user will take supplements and/or vitamins, use other health-conscious products, implement and/or vary an exercise regimen, etc. For example, in instances in which the processing logic determines, from the personal health data being compared to the predictive model, the user has an increased risk in heart health, the processing logic may determine the user is more likely to use heart healthy supplements, increase exercise, and/or other actions to improve heart health. The processing logic may determine additional predictive models based on one or more organs and/or organ systems using any number of user health indicators, such as biomarker data and/or longitudinal biomarker data.

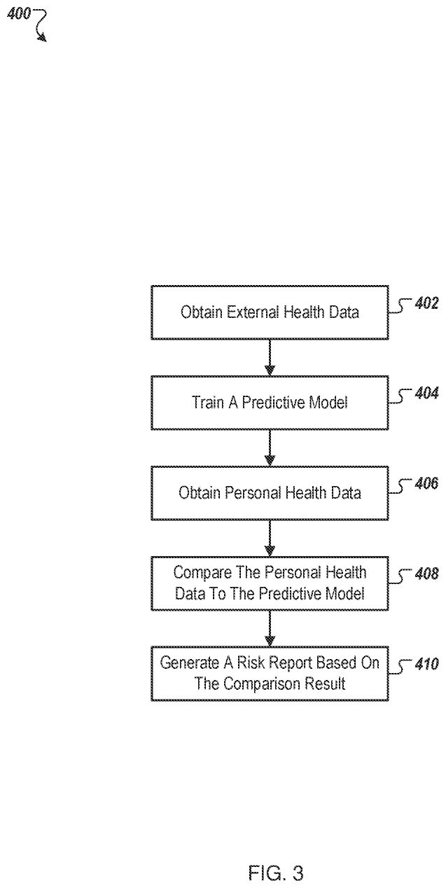

FIG. 3 illustrates a flowchart of an example method 400 of generating a risk report, in accordance with at least one embodiment described in the present disclosure. In some embodiments, generating the risk report may include training a predictive model based on external health data and comparing personal health data to the predictive model. The method 400 may be performed by any suitable system, apparatus, or device). For example, the health application 111 of FIG. 1, the second system 140 of FIG. 1 (e.g., as directed by the health application 111) may perform or direct performance of one or more of the operations associated with the method 400. The method 400 may include one or more blocks 402, 404, 406, 408, or 410. Although illustrated with discrete blocks, the steps and operations associated with one or more of the blocks of the method 400 may be divided into additional blocks, combined into fewer blocks, or eliminated, depending on the particular implementation.

At block 402, external health data may be obtained. The external health data may correspond to other people. For example, the health application 111 of FIG. 1 may obtain the external health data.

At block 404, a predictive model may be trained. The predictive model may be trained using the external health data. The predictive model may generate associations between personal health data related to a user and the external health data. For example, the health application 111 of FIG. 1 may train the predictive model using the external health data to generate the associations between the personal health data and the external health data.

At block 406, the personal health data may be obtained. The personal health data may relate to the user. For example, the health application 111 of FIG. 1 may obtain the personal health data.

At block 408, the personal health data may be compared to the predictive model. For example, the health application 111 may compare the personal health data to the predictive model to generate associations between the personal health data and potential health risk of the user.

At block 410, a risk report may be generated based on the comparison result. The risk report may indicate the potential health risk of the user, a mitigation recommendation, or some combination thereof.

Modifications, additions, or omissions may be made to the method 400 without departing from the scope of the present disclosure. For example, the operations of method 400 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

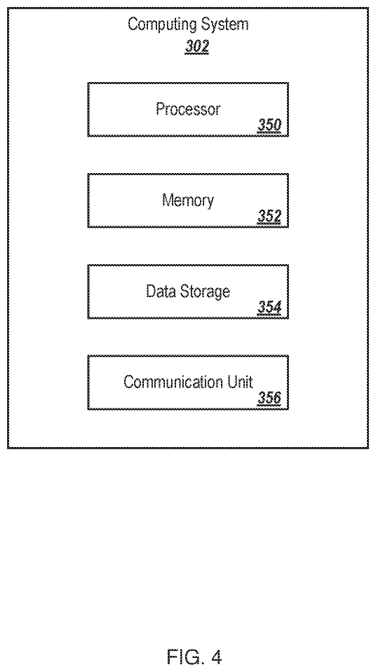

FIG. 4 illustrates a block diagram of an example computing system 302, according to at least one embodiment of the present disclosure. The computing system 302 may be configured to implement or direct one or more operations associated with a system and/or a network (e.g., the first system 130, the second system 140 and/or the network 105 of FIG. 1). The computing system 302 may include a processor 350, a memory 352, a data storage 354, and a communication unit 356. The processor 350, the memory 352, the data storage 354, and the communication unit 356 may be communicatively coupled.

In general, the processor 350 may include any suitable special-purpose or general-purpose computer, computing entity, or processing device including various computer hardware or software modules and may be configured to execute instructions stored on any applicable computer-readable storage media. For example, the processor 350 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 350 may include any number of processors configured to, individually or collectively, perform or direct performance of any number of operations described in the present disclosure. Additionally, one or more of the processors may be present on one or more different electronic devices, such as different servers.

In some embodiments, the processor 350 may be configured to interpret and/or execute program instructions and/or process data stored in the memory 352, the data storage 354, or the memory 352 and the data storage 354. In some embodiments, the processor 350 may fetch program instructions from the data storage 354 and load the program instructions in the memory 352. After the program instructions are loaded into memory 352, the processor 350 may execute the program instructions.

For example, in some embodiments, the modification module may be included in the data storage 354 as program instructions. The processor 350 may fetch the program instructions of a corresponding module from the data storage 354 and may load the program instructions of the corresponding module in the memory 352. After the program instructions of the corresponding module are loaded into memory 352, the processor 350 may execute the program instructions such that the computing system may implement the operations associated with the corresponding module as directed by the instructions.

The memory 352 and the data storage 354 may include computer-readable storage media for carrying or having computer-executable instructions or data structures stored thereon. Such computer-readable storage media may include any available media that may be accessed by a general-purpose or special-purpose computer, such as the processor 350. By way of example, and not limitation, such computer-readable storage media may include tangible or non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 350 to perform a certain operation or group of operations.

The communication unit 356 may include any component, device, system, or combination thereof that is configured to transmit or receive information over a network. In some embodiments, the communication unit 356 may communicate with other devices at other locations, the same location, or even other components within the same system. For example, the communication unit 356 may include a modem, a network card (wireless or wired), an infrared communication device, a wireless communication device (such as an antenna), and/or chipset (such as a Bluetooth® device, an 802.6 device (e.g., Metropolitan Area Network (MAN)), a WiFi device, a WiMax device, cellular communication facilities, etc.), and/or the like. The communication unit 356 may permit data to be exchanged with a network and/or any other devices or systems described in the present disclosure. For example, when the computing system 302 is included in the first user device 110 of FIG. 1, the communication unit 356 may allow the first user device 110 to communicate with the first system 130 and/or the second system 140, via the network 105.

Modifications, additions, or omissions may be made to the computing system 302 without departing from the scope of the present disclosure. For example, in some embodiments, the computing system 302 may include any number of other components that may not be explicitly illustrated or described.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method comprising:
obtaining external health data corresponding to other people;
training a predictive model using the external health data the predictive model generates an association between personal health data related to a user and the external health data;
obtaining the personal health data;
comparing the personal health data to the predictive model; and
generating a risk report based on the comparison, the risk report indicating a potential health risk of the user.

2. The method of claim 1, wherein:
the user comprises a group of people and the personal health data corresponds to each person within the group of people; and
the risk report indicates the potential health risk of each person within the group of people or all people of the group of people.

3. The method of claim 2 further comprising:
determining the potential health risk of the group of people based on the comparison of the personal health data to the predictive model based on biomarker data within the personal health data exceeding a threshold value in the predictive model;
determining a mitigation recommendation to mitigate the potential health risk of the group of people, wherein:
the risk report comprises the potential health risk and the mitigation recommendation; and
the mitigation recommendation indicates at least one of a suggested change to an environment of the group of people, schedule of breaks during a period of time for the group of people to exercise, or schedule of food to consume that improves a specific aspect of a health of the group of people.

4. The method of claim 1 further comprising determining a risk metric indicating a likelihood that the user will experience health issues at a future time.

5. The method of claim 1, wherein:
the personal health data comprises at least one of a biomarker data, a longitudinal biomarker data, a weight, a height, a resting heart rate, a gender, a waist circumference, a blood pressure, a cholesterol level, a blood sugar level, organ context data, or organ system context data related to the user; and
the personal health data indicates an association between at least two of the biomarker data, the longitudinal biomarker data, the weight, the height, the resting heart rate, the gender, the waist circumference, the blood pressure, the cholesterol level, the blood sugar level, the organ context data, or the organ system context data related to the user.

6. The method of claim 1 further comprising:
determining the potential health risk of the user based on the comparison of the personal health data to the predictive model based on biomarker data within the personal health data exceeding a threshold value in the predictive model; and
determining a mitigation recommendation to mitigate the potential health risk, wherein the risk report comprises the potential health risk and the mitigation recommendation.

7. The method of claim 1 further comprising:
receiving organ context data representative of a health status of an organ or an organ system of the user; and
associating the personal health data and the organ context data, wherein the comparing the personal health data to the predictive model is further based on the association between the personal health data and the organ context data.

8. A non-transitory computer-readable medium having computer-readable instructions stored thereon that are executable by a processor to perform or control performance of operations comprising:
obtaining external health data corresponding to other people;
training a predictive model using the external health data the predictive model generates an association between personal health data related to a user and the external health data;
obtaining the personal health data;
comparing the personal health data to the predictive model; and
generating a risk report based on the comparison, the risk report indicating a potential health risk of the user.

9. The non-transitory computer-readable medium of claim 8, wherein:
the user comprises a group of people and the personal health data corresponds to each person within the group of people; and
the risk report indicates the potential health risk of each person within the group of people or all people of the group of people.

10. The non-transitory computer-readable medium of claim 9, wherein the operations further comprise:
determining the potential health risk of the group of people based on the comparison of the personal health data to the predictive model based on biomarker data within the personal health data exceeding a threshold value in the predictive model;
determining a mitigation recommendation to mitigate the potential health risk of the group of people, wherein:
the risk report comprises the potential health risk and the mitigation recommendation; and
the mitigation recommendation indicates at least one of a suggested change to an environment of the group of people, schedule of breaks during a period of time for the group of people to exercise, or schedule of food to consume that improves a specific aspect of a health of the group of people.

11. The non-transitory computer-readable medium of claim 8, wherein the operations further comprise determining a risk metric indicating a likelihood that the user will experience health issues at a future time.

12. The non-transitory computer-readable medium of claim 8, wherein:
the personal health data comprises at least one of a biomarker data, a longitudinal biomarker data, a weight, a height, a resting heart rate, a gender, a waist circumference, a blood pressure, a cholesterol level, a blood sugar level, organ context data, or organ system context data related to the user; and
the personal health data indicates an association between at least two of the biomarker data, the longitudinal biomarker data, the weight, the height, the resting heart rate, the gender, the waist circumference, the blood pressure, the cholesterol level, the blood sugar level, the organ context data, or the organ system context data related to the user.

13. The non-transitory computer-readable medium of claim 8, wherein the operations further comprise:
determining the potential health risk of the user based on the comparison of the personal health data to the predictive model based on biomarker data within the personal health data exceeding a threshold value in the predictive model; and
determining a mitigation recommendation to mitigate the potential health risk, wherein the risk report comprises the potential health risk and the mitigation recommendation.

14. The non-transitory computer-readable medium of claim 8, wherein the operations further comprise:
receiving organ context data representative of a health status of an organ or an organ system of the user; and
associating the personal health data and the organ context data, wherein the operation comparing the personal health data to the predictive model is further based on the association between the personal health data and the organ context data.

15. A system comprising:
a computer-readable storage media configured to store instructions; and
a processor operatively coupled to the computer-readable storage media and configured to cause the system to perform operations comprising:
obtain external health data corresponding to other people;
train a predictive model using the external health data the predictive model generates an association between personal health data related to a user and the external health data;
obtain the personal health data;
compare the personal health data to the predictive model; and
generate a risk report based on the comparison, the risk report indicating a potential health risk of the user.

16. The system of claim 15, wherein:
the user comprises a group of people and the personal health data corresponds to each person within the group of people; and
the risk report indicates the potential health risk of each person within the group of people or all people of the group of people.

17. The system of claim 16, wherein the operations further comprise:
determine the potential health risk of the group of people based on the comparison of the personal health data to the predictive model based on biomarker data within the personal health data exceeding a threshold value in the predictive model;
determine a mitigation recommendation to mitigate the potential health risk of the group of people, wherein:
the risk report comprises the potential health risk and the mitigation recommendation; and
the mitigation recommendation indicates at least one of a suggested change to an environment of the group of people, schedule of breaks during a period of time for the group of people to exercise, or schedule of food to consume that improves a specific aspect of a health of the group of people.

18. The system of claim 15, wherein:
the personal health data comprises at least one of a biomarker data, a longitudinal biomarker data, a weight, a height, a resting heart rate, a gender, a waist circumference, a blood pressure, a cholesterol level, a blood sugar level, organ context data, or organ system context data related to the user; and
the personal health data indicates an association between at least two of the biomarker data, the longitudinal biomarker data, the weight, the height, the resting heart rate, the gender, the waist circumference, the blood pressure, the cholesterol level, the blood sugar level, the organ context data, or the organ system context data related to the user.

19. The system of claim 15, wherein the operations further comprise:
determine the potential health risk of the user based on the comparison of the personal health data to the predictive model based on biomarker data within the personal health data exceeding a threshold value in the predictive model; and
determine a mitigation recommendation to mitigate the potential health risk, wherein the risk report comprises the potential health risk and the mitigation recommendation.

20. The system of claim 15, wherein the operations further comprise:
receive organ context data representative of a health status of an organ or an organ system of the user; and
associate the personal health data and the organ context data, wherein the operation compare the personal health data to the predictive model is further based on the association between the personal health data and the organ context data.
